(19) United States
(12) Patent Application Publication
(10) Pub. No.: US 2007/0239202 A1
(43) Pub. Date: Oct. 11, 2007

(54) Abrasively coated surgical end effector

(76) Inventor: Richard A. Rodriguez, Arvada, CO (US)

Correspondence Address: Richard A. Rodriguez, 10961 West 68th Place, Arvada, CO, 80004, US

(21) Appl. No.: 11/400,830

(22) Filed: Apr. 10, 2006

(51) Int. Cl. A61B 17/00

(52) U.S. Cl. 606/205

(57) ABSTRACT

A surgical end effector having a top edge, outer surfaces, and gripping surfaces, with an air entrapment enhancing abrasive coating upon the outer surfaces.

This invention relates to a surgical instrument such as a forceps, scissors or scalpel blade, and to coatings thereupon.

In particular, the invention relates to surgical end effectors incorporating an abrasive outer coating. End effectors may take the form of scissors, grippers, cutting jaws, forceps, scalpels and the like. The invention has particular use with respect to endoscopic or laparoscopic surgical processes. Various types of endoscopic surgical instruments are known in the art. For clarity, the term “endoscopic instruments” includes laparoscopic, arthroscopic, and neurological instruments, as well as instruments which are inserted through an endoscope. Such instruments are commonly used in surgical intervention, most commonly in “keyhole” or minimally invasive surgery, but also in “open” surgery. For minimal tissue damage such ports are made as small as possible with typical port sizes being in the range of from 5 mm to 12.5 mm.

### SUMMARY OF THE INVENTION

A primary embodiment of the invention comprises a surgical instrument in the form of forceps jaws, having a substrate made of metal, together comprising a pair of pivotally mounted metallic jaws having a top edge, outermost faces, and a toothed or smooth gripping surface. The forceps jaws can be fabricated from any of a number of materials that include metals, ceramics, or plastic. The jaws have an outer layer of an abrasive coating upon the top edge and outermost faces of the forceps jaws including the gripping surface. The produced jaw according to the present invention has a high degree of hardness and toughness, is resistant to corrosion and includes an abrasive outer coating that is well adhered. The abrasive coating preferably is a self bonding semi fused plasma coating.

The coating process may include gritblasting, or depositing a metallic material on the jaw by deposition to form a first coated layer on the surface. This depositing step can also add a higher grit abrasive compound built upon the first coated layer using a process to form a second coating layer.

Because of their very small size and the requirements of strength and/or sharpness, end effectors are difficult to manufacture and are typically formed of forged stainless steel, and are an expensive portion of the endoscopic instrument. Forceps made of metal are common and their manufacture is generally costly since the part which performs the gripping action has to be precision finished. If the forceps are mass-produced their quality is generally inferior especially as far as accuracy of the gripping part is concerned which must contain grooves and teeth in order to able to grip veins, delicate threads, hair, body tissue and the like. Additionally, there are plastic forceps produced, where the teeth and grooves of the gripping parts can be manufactured accurately by molding. However, these forceps are not very strong and are prone to bend during use.

A problem arises when tissue or blood adheres to the surgical forceps, interfering with the performance of the surgeon and shortening the life of the instrument. Various non-stick coatings have been developed to try to alleviate this problem, the most common material for such coatings being fluoropolymers or similar.

The present instrument is of particular advantage when parting or moving tissue aside during gall bladder surgery. This is a difficult operation when performed remotely and it is advantageous to be able to perform the procedure quickly, and use the instrument without it sticking to adjacent tissue structures during the procedure. Additionally, the present invention provides a non light-reflective forceps jaw surface, which helps to reduce lighting glare experienced by prior glossy surfaced surgical forceps, while magnified in close proximity to a light source, as is commonly encountered in many laparoscopic procedures.

It is an object of the present invention to provide a surgical forceps which combines the advantages of both metal and plastic, along with the improved function of an abrasively coated outer jaw portion which diminishes tissue adherence during surgical processes. In general, the particle size of a coating material can influence the texture of the coatings, with larger particles tending to produce a rougher surface texture than smaller particles. In the present invention, the use of a rougher surface texture results in better tissue release characteristics, due to enhanced air entrapment and reduced suction along the forceps jaw as it is presented adjacent to the tissue.

The preferred coating material, tungsten carbide appears to be a suitable coating material. However, coatings of tungsten carbide are not as adherent to some metallic alloys as are coatings like Armacor M, and the typical size of the tungsten carbide particles deposited are usually much smaller than particles of the Armacor M material. Titanium nitride, diamond, or titanium carbide coatings could also be used, as well as simple gritblasting.

### PRIOR ART

To reduce the adhesion of blood and tissue, and accumulations upon surgical instruments, many coatings have been tried in the past. Additionally, differing coatings have been applied to surgical tools for other diverse purposes. Included among these prior surgical implement coatings are:

The electrosurgical instrument of U.S. Pat. No. 6,966,909 to Marshall et al, that discloses a tungsten disulphide coating that minimizes tissue adhesion upon the electrode surface during electrocauterization;

The self-sacrificing intraocular tool coating presented in U.S. Pat. No. 4,170,043 to Knight et al, disclosing the biocompatible water soluble adherent film coating that lasts for less than 24 hours when placed in an aqueous medium;

The bipolar coated endoscopic scissor blades shown in U.S. Pat. No. 5,893,846 to Bales et al, disclosing a method for application of a ceramic coating that minimizes tissue adhesion upon bipolar electrocautery scissors and serves as electrical insulation;

The coated blade described in U.S. Pat. No. 5,120,596 to Yamada, that illustrates a titanium ceramic surface coated blade which improves both sharpness and corrosion resistance;

The dentists forceps of U.S. Pat. No. 6,345,983 to Godfrey that provides for a diamond coating on the tips of the dental forceps to enhance grip upon the metal matrix surrounding a tooth during tooth filling;

The sandwiched composite medical forceps or clamp shown in U.S. Pat. No. 4,727,876 to Porat, et al illustrating provision of a molded on plastic over-layer coating which serves as the tissue grip or clamp surface;

The wear resistant medical/dental pliers disclosed in U.S. Pat. No. 5,257,558 to Farzin-Nia et al which are coated with a thickened self-supportive coating which helps to prolong the life of the instrument.

The present invention provides an alternative and an improvement to such prior coatings, in terms of its ability to resist the adhesion of adjacent tissue during standard or laparoscopic surgical use. In the present invention, the use of a rough surface texture upon the forceps outer faces results in excellent tissue release characteristics, due to enhanced air entrapment along the forceps jaw presented adjacent to the tissue. During prototype test use of the present invention the surgeon is able to accurately and more quickly navigate amongst sticky muscle and organ tissue because the present invention forceps do not easily permit the adjacent tissue to reposition itself in relation to the tool surface during use, reducing procedure time by 20-25 minutes.

Additionally, the present invention provides for a non light-reflective forceps jaw surface, as the resultant color of the forceps jaw coating is matte gray, which helps to reduce lighting glare experienced by prior glossy surfaced forceps, while in close proximity to a light source, as is commonly encountered in many laparoscopic procedures.

### BRIEF DRAWINGS DESCRIPTION

The invention will now be described with reference to the drawings in which:

FIG. 1 is a plan view of the forceps jaws, shown with open jaws,

FIG. 2 is a side view of the closed forceps jaws

FIG. 3 is a magnified sectional view of the forceps outer surface coating.

### DETAILED DRAWINGS DESCRIPTION

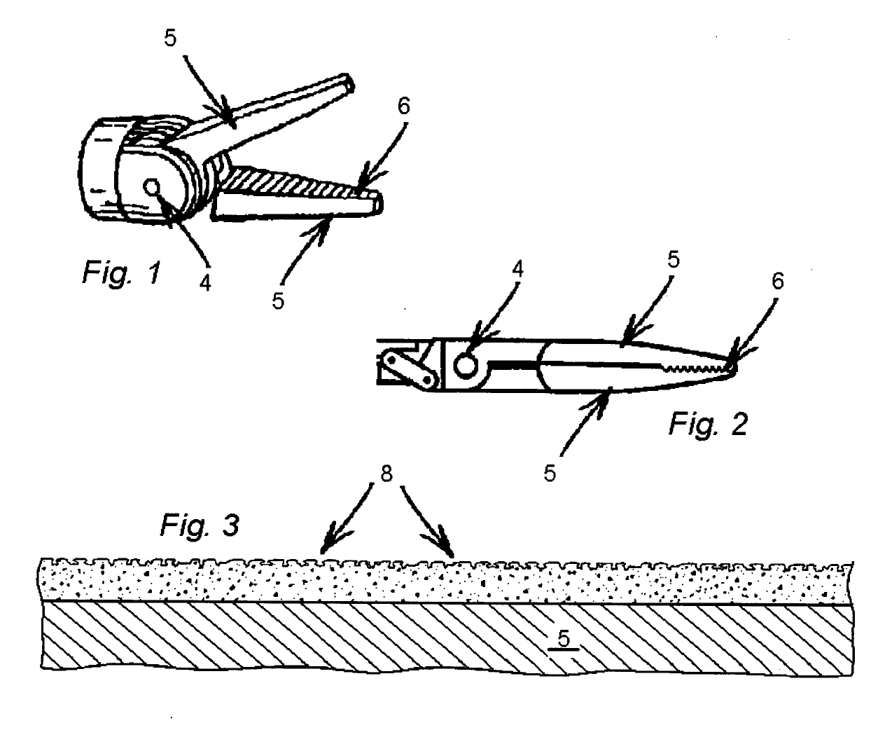

A preferred embodiment of the invention now to be described is a pair of hinged jaw 5 bodies, together serving as medical forceps capable of being used for laparoscopic, endoscopic, arthroscopic or open surgery. Referring to FIGS. 1 to 3 of the drawings, the improved medical instrument, is here first presented in the form of a pair of pivotally 4 mounted stainless steel metallic forceps jaw 5 bodies, each having a top edge, outermost faces, and a toothed or smooth gripping surface 6. Each jaw 5 body is comprised of a single metal component of stainless steel. Each jaw 5 body comprises a distally tapered body, with a hinge 4 point at its proximal end. However, the forceps jaw 5 according to the invention can be made in the shape and material of any previously known forceps. Each jaw 5 is pivoted at its proximal end on the hinge 4 point and is pivotable between open and closed positions. In FIG. 1 the jaws 5 are shown in their open position. In FIG. 2 the jaws 5 are shown in their closed position. Referring to FIG. 1 and FIG. 2, each jaw 5 body is coated with an abrasive coating compound 8 such as a self-bonding, semi fused, self-fluxing alloy, applied upon substantially all surfaces, including the gripping surface 6. The abrasive coating 8 prevents the forceps jaw 5 adherence to tissue adjacent the operation site and improves surgeon navigation past the body tissue. If desired, the abrasive coating 8 may cover the entire surface of the forceps jaw 5, or may be placed over only a part of the forceps jaw 5 which is less than that above described. Further, the forceps jaw 5 may be provided with a smoothed gripping part 6 without teeth or grooves. As presented here in FIG. 3, the particle size of the coating 8 material being deposited can influence the texture of the forceps jaw 5 outer surfaces with larger particles which produce a rougher outer surface texture than smaller particles. To a degree, a rougher surface texture results in better tissue release characteristics, due to enhanced air entrapment and reduced suction along the forceps jaw 5 presented adjacent to the tissue during surgical procedures.

During the preceding detailed description, there has been disclosed and illustrated several general principles and methods of abrasively coating and providing surface texturing of endoscopic surgical end effectors. While particular embodiments of the invention have been described, it is not intended that the invention be limited thereto, as it is intended that the invention be as broad in scope as the art will allow and that the specification be read likewise.

Thus, while application of a particular abrasive surface texture has been disclosed, it will be appreciated that other levels of texturing more or less irregular than that disclosed can be used as well. Also while a particular surgical instrument incorporating the textured abrasive coating has been herein described, it will be appreciated by those skilled in the art that other surgical instruments, and end effectors, whether manual standard, non-cautery, monopolar or bipolar, and the use of other actuating mechanisms, clamps or jaw types can be abrasively coated. Furthermore, while particular methods for roughening the jaw to provide for abrasive coating and applications thereof were described, it will be appreciated that other methods could be utilized. It will therefore be appreciated by those skilled in the art that yet other modifications could be made to the provided invention without deviating from its spirit and scope as so claimed.

What is claimed is:

1. An improved surgical forceps instrument for the manipulation of tissue, including one or more outer surfaces likely to contact internal tissue, said outer surfaces comprising a plurality of jaw portions, with at least one jaw portion manufactured from a stainless steel or titanium metallic substrate, each said jaw portion having a grasping or holding surface; and;
Application of an abrasive coating on said outer surfaces by physical deposition of an alloy material selected from the group consisting of tungsten, carbide, nickel, titanium, zirconium, silicon, or diamond, whereby the coated portion of the instrument has a textured, micro abrasive outer surface.

2. A surgical instrument according to claim 1, wherein the instrument is a surgical end effector type forceps instrument.

3. A surgical instrument according to claim 1, wherein the abrasive coating is a spray deposited coating.

4. A surgical instrument according to claim 1, wherein the abrasive coating is a plasma deposited coating.

5. A surgical instrument according to claim 1, wherein the instrument is provided with an abrasive coating having a thickness of from about one thousandth to about one twenty-thousandth of an inch.

6. A surgical instrument as set forth in claim 1, wherein only a portion of the instrument is abrasively coated.

7. A surgical instrument as set forth in claim 1, wherein the coating includes a plurality of applied abrasive coating layers.

8. The surgical instrument of claim 1 wherein said abrasive coating is formed from a multiplicity of applied particles of said abrasive material.

9. A surgical instrument according to claim 1 with a textured outer surface provided by formulating the metal substrate material with the abrasive component included within the substrate material.

10. The surgical instrument of claim 1, wherein said outer surfaces are pre textured before said coating is applied.

11. The surgical instrument of claim 1, where said grasping faces are smooth.

12. The surgical instrument of claim 1, where said grasping faces are toothed.

13. A surgical instrument according to claim 1, where said textured surface has a roughness approximately at least as rough as a roughness of a grit blasted instrument.

14. A surgical instrument as recited in claim 1, wherein said depositing step further comprises the step of forming a compound on said first coated abrasive layer using a chemical reaction process to form a second coated layer.

15. The surgical instrument of claim 1, wherein said micro abrasive material is Armacor M.

16. A surgical instrument according to claim 1, wherein said jaw portions are manufactured of a thermoplastic material.

17. The surgical instrument as recited in claim 1, wherein said jaw portions are manufactured of a ceramic material.

18. A surgical instrument according to claim 1, wherein said jaw portions are manufactured of a composite material.

19. A method for manufacturing a stainless steel, composite, or titanium jawed surgical instrument for the manipulation of tissue, said instrument constituting a pair of jaws tapering distally from a pivot axis, at least one of the jaws being pivotally mounted to allow pivotal opening and closing movements with respect to the other jaw about said pivot axis, the method including the steps of manufacturing at least one jaw portion from any of said stainless steel, composite, or titanium substrates, each jaw portion having an integral grasping or holding surface, and disposing a plasma process coating of semi fused tungsten carbide nickel based self fluxing alloy fully on to said jaws, subsequently grinding said coating to a desired roughness sufficient to provide a micro abrasive characteristic upon said jaws.

20. A surgical instrument according to claim 2 with a textured outer surface provided by formulating the metal substrate material with the abrasive component included within the substrate material.
